(19) United States
(12) Patent Application Publication
(10) Pub. No.: US 2020/0097651 A1
(43) Pub. Date: Mar. 26, 2020

(54) SYSTEMS AND METHODS TO ACHIEVE ROBUSTNESS AND SECURITY IN MEDICAL DEVICES

(71) Applicant: General Electric Company, Schenectady, NY (US)

(72) Inventors: Lalit Keshav MESTHA, North Colonie, NY (US); Hema ACHANTA, Niskayuna, NY (US); Olugbenga ANUBI, Niskayuna, NY (US)

(21) Appl. No.: 16/142,841

(22) Filed: Sep. 26, 2018

(51) Int. Cl. G06F 21/55; G16H 40/63

(52) U.S. Cl. CPC G06F 21/554; G06F 2221/034; G16H 40/63

(57) ABSTRACT

According to some embodiments, a system, method and non-transitory computer-readable medium are provided comprising one or more heterogeneous data source nodes generating data associated with operation of the medical device; an abnormal state detection, prediction and correction module to receive data from one or more heterogeneous data source nodes; a memory for storing program instructions; and an abnormal state processor, coupled to the memory, and in communication with the abnormal state detection, prediction and correction module and operative to execute program instructions to: receive data from one or more heterogeneous data source nodes; receive a decision manifold separating a normal operating space from an abnormal operating space; perform a feature extraction process on the received data to generate at least one feature vector; determine, via the abnormal state detection, prediction and correction module, whether the feature vector maps to the normal operating space or the abnormal operating space in the decision manifold; and generate, via the abnormal state detection, prediction and correction module, a corrected value for the feature vector to map the feature vector to the normal operating space when it is determined that the feature vector maps to the abnormal operating space. Numerous other aspects are provided.

### BACKGROUND

Medical devices that are used by patients may communicate with elements external to the device. As a result, control systems associated with the medical devices may be vulnerable to threats, such as cyber-attacks (e.g., associated with a computer virus, malicious software, etc.), or device malfunctioning that could disrupt the operation of the medical device. Current methods of protection from this type of harm primarily consider threat detection via acoustic signals.

It would be desirable to provide systems and methods to improve security of medical devices in an automatic and accurate manner.

### SUMMARY

According to some embodiments, a system to protect a medical device includes one or more heterogeneous data source nodes generating data associated with operation of the medical device; an abnormal state detection, prediction and correction module to receive data from one or more heterogeneous data source nodes; a memory for storing program instructions; and an abnormal state processor, coupled to the memory, and in communication with the abnormal state detection, prediction and correction module and operative to execute program instructions to: receive data from one or more heterogeneous data source nodes; receive a decision manifold separating a normal operating space from an abnormal operating space; perform a feature extraction process on the received data to generate at least one feature vector; determine, via the abnormal state detection, prediction and correction module, whether the feature vector maps to the normal operating space or the abnormal operating space in the decision manifold; and generate, via the abnormal state detection, prediction and correction module, a corrected value for the feature vector to map the feature vector to the normal operating space when it is determined that the feature vector maps to the abnormal operating space.

According to some embodiments, a computer-implemented method to protect a medical device includes receiving data from one or more heterogeneous data source nodes; receiving a decision manifold separating a normal operating space from an abnormal operating space; performing a feature extraction process on the received data to generate at least one feature vector; determining, via an abnormal state detection, prediction and correction module, whether the feature vector maps to the normal operating space or the abnormal operating space in the decision manifold; and generating, via the abnormal state detection, prediction and correction module, a corrected value for the feature vector to map the feature vector to the normal operating space when it is determined that the feature vector maps to the abnormal operating space.

According to some embodiments, a non-transitory computer-readable medium storing instructions that, when executed by a computer processor, cause the computer processor to perform a method including receiving data from one or more heterogeneous data source nodes; receiving a decision manifold separating a normal operating space from an abnormal operating space; performing a feature extraction process on the received data to generate at least one feature vector; determining, via an abnormal state detection, prediction and correction module, whether the feature vector maps to the normal operating space or the abnormal operating space in the decision manifold; and generating, via the abnormal state detection, prediction and correction module, a corrected value for the feature vector to map the feature vector to the normal operating space when it is determined that the feature vector maps to the abnormal operating space.

Some technical effects of some embodiments disclosed herein are improved systems and methods to protect a medical device from malicious intent such as cyber-attacks, and from device malfunctions, in an automatic and accurate manner. Another technical effect of some embodiments is that the protection is via the neutralization (i.e. correction) of the effects of the abnormalities in the operation of the device in situ. Some embodiments provide for the use of Multi-Modal, Multi-Disciplinary (MMMD) features containing bio-electromechanical physics of medical devices, human physiology, fluid dynamics (e.g., hemodynamics) and machine learning using local and global features. A technical effect of using these MMMD features is that medical device behavior may be captured, as well as the physiological state of patients. A technical effect of some embodiments is the provision of self-defense (e.g., neutralization) processes operating in conjunction with detection and forecasting, and in particular for LVAD and medical device operations. Some embodiments provide for the continued operation of the device through these “self-defense” processes, which may save the patient's life. It is noted that the remote monitoring of pump parameters and hemodynamics with real-time communication between caregiver, patients, and device, as providing by some embodiments may save life and improve patient outcome.

The inventors note that although echocardiogram (ECG) ramp studies may be used to diagnose pump faults such as thrombosis, such technology cannot be used easily for in-home settings. Some embodiments for detecting faults may achieve sensitive and specific detection of key faults, such as thrombosis, and may be easily incorporated for use in home setting environments.

### BRIEF DESCRIPTION OF THE DRAWINGS

FIG. 1 is a high-level block diagram of a system that may be provided in accordance with some embodiments.

FIG. 2 is a method according to some embodiments.

FIG. 3 is an abnormal alert system in accordance with some embodiments.

FIG. 4 illustrates boundaries and a feature vector for a medical device parameter according to some embodiments.

FIG. 5 is an offline and real-time anomaly decision and early warning tool architecture according to some embodiments.

FIG. 6 illustrates a feature vector information flow diagram in accordance with some embodiments.

FIG. 7 is a feature vector information flow diagram for a non-exhaustive example in accordance with some embodiments.

FIG. 8 is a method for creating a selected feature subset according to some embodiments.

FIG. 9 is a block diagram of a medical device protection platform according to some embodiments of the present invention.

FIG. 10 is a tabular portion of a medical device database in accordance with some embodiments.

FIG. 11 is a tabular portion of data source database in accordance with some embodiments.

FIG. 12 is a tabular portion of an alert database according to some embodiments.

FIG. 13 is a display according to some embodiments.

### DETAILED DESCRIPTION

In the following detailed description, numerous specific details are set forth in order to provide a thorough understanding of embodiments. However it will be understood by those of ordinary skill in the art that the embodiments may be practiced without these specific details. In other instances, well-known methods, procedures, components and circuits have not been described in detail so as not to obscure the embodiments.

Medical devices that are used by patients for monitoring/maintaining bodily operations may communicate with elements external to the device. As a non-exhaustive example, in the United States, 6.5 million adults live with heart failure, and more than 70,000 patients have advanced heart failure requiring a heart transplant. While the number of patients awaiting a transplant has doubled in the last fifteen years, for example, the worldwide availability of donor hearts has decreased by a third, thus necessitating the use of mechanical assist devices (e.g., left ventricular assist devices (“LVADs”). Although LVADs are incredibly durable, failures may occur. LVADs, for example, fail in nearly one out of six patients due to pump thrombosis (i.e., faults), leading to additional surgeries, complications, and even death. The most common cause of failure may be due to pump thrombosis, the formation of a blood clot at an interface between blood and the device. Exacerbated thrombosis may result in hemodynamic derangement, stroke, and death. Additionally, medical devices may engage in bi-/uni-directional communication (e.g., wireless communication), which may allow physicians to monitor and potentially adjust device parameters remotely, based on sensed measurements. However, when medical devices, such as LVADs, engage in bi-/uni-directional communication, they may be prone to perturbations outside the normal operating space. For example, hackers may gain illegal access to the medical device and intentionally manipulate some aspect (e.g., the pump power, drain the battery, slowly vary flow rate inducing, in the case of the LVAD a stealthy thrombus attack, or completely stop) of the device, which may cause damage or death.

Embodiments provide for detecting when a perturbation event has happened or is about to happen (i.e. forecasting) and then neutralizing the effects of the likely perturbation in real-time. One or more embodiments provide for neutralizing the effects of abnormalities in the operation of the device so that the device may be capable of “self-defense” in the presence of faults or cyber-attacks for continued operation, ensuring patient safety. For example, if an attacker maliciously changes the pump speed signal in a LVAD, the attack may dramatically change the operation of the LVAD, moving the device operation to abnormal operating space. The LVAD, or any other medical device, may include a controller to control the operation of the device to operate the device at a normal state based on signals received from the device and rules programmed into the controller. Device action is dependent on the signal it receives from the controller. In terms of an attack, the mal-doer may try to attack the signal between the controller and the device, so that the controller is tricked into making a harmful decision.

One or more embodiments provide for the detection or anticipation of this abnormality using decision manifolds (and forecasting in some instances), and may correct signal input to controller of the medical device to operate the medical device at a healthy/correct pump speed, for example, or any other “normal” operation. One or more embodiments provide for intercepting the signal prior to receipt by the controller to determining if it's abnormal, such that the controller may only receive correct signals. An abnormal state detection, prediction and correction module may receive the raw intercepted data signals and transform them to a higher or lower dimensional feature space, depending on the machine learning process used, and then may determine if features associated with the signals are in the normal or abnormal space. An optimization strategy may then be executed in one or more embodiments, to maintain an optimum (normal) operating point or to move the operating point back to the optimum/normal state from the abnormal state.

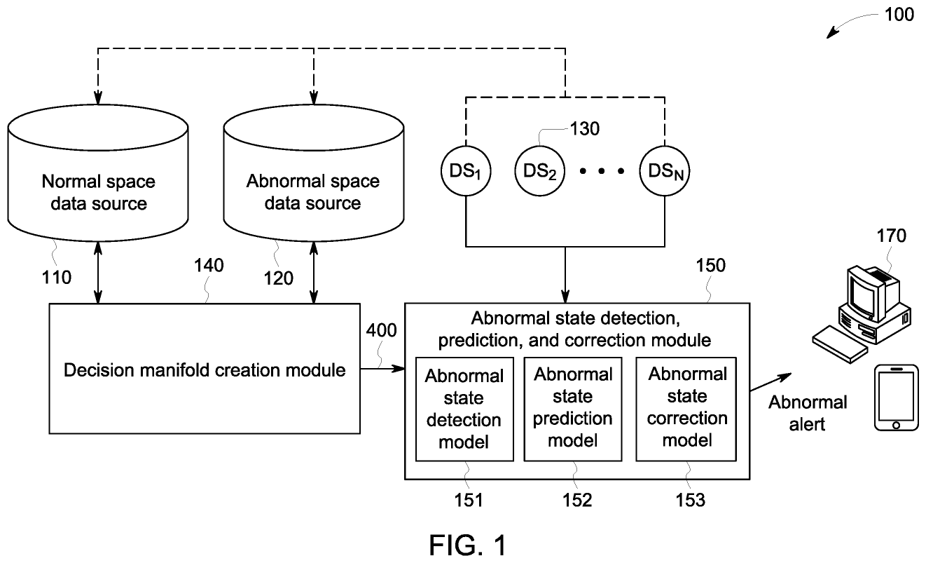

FIG. 1 is a high-level architecture of a system 100 in accordance with some embodiments. In one or more embodiments, the system may be located on the medical device (e.g. part of the controller, in a chip coupled to the controller), or in a cloud associated with the medical device. The system 100 may include a “normal space” data source 110 and an “abnormal space” data source 120. The normal space data source 110 and the abnormal data source 120 might store, for each of a plurality of heterogeneous “data source nodes” 130 (shown in FIG. 1 as “DS₁”, “DS₂,” “DS_{N}” for “1, 2, . . . N” different data source nodes), a series of normal values over time that represent normal operation of a medical device (e.g., generated by a model or collected from actual data source node 130 data as illustrated by the dashed line in FIG. 1). As used herein, the phrase “data source node” might refer to, for example, sensor data, physics-based models, data-driven models, and patient/user inputs. The nodes may receive data from other aspects of the system in a continuous fashion in the form of continuous signals or streams of data or combinations thereof. Moreover, the nodes 130 may be used to monitor occurrences of cyber-threats or abnormal events.

The abnormal space data source 120 might store, for each of the data source nodes 130, a series of abnormal values that represent an abnormal operation of the medical device (e.g., when the system is experiencing a cyber-attack or fault). In one or more embodiments, physics-based and other models may be used to generate “abnormal” data (“training data sets”). For example, attack signatures may be induced on the data and then the resulting effects on the data may be analyzed to determine how faults affect the data versus attacks affect the data. According to some embodiments, the data source nodes 130 provide “heterogeneous” data. That is, the data may represent information from widely diverse areas, such as acoustic sensors, patient/user inputs, models, etc.

Information from the normal space data source 110 and the abnormal space data source 120 may be provided to an offline decision manifold creation module 140 that uses this data to create a decision boundary (that is, a boundary that separates normal behavior from abnormal behavior). It is noted that while an offline decision manifold may be described herein, one or more embodiments may use a computing online decision manifold. The decision boundary may then be used by an abnormal state detection model 151, a prediction model 152 and a correction model 153. The abnormal state detection, prediction and correction module 150 may, for example, monitor streams of data from the data source nodes 130 comprising data from sensor nodes, and/or any other critical data source nodes (e.g., data source nodes DS₁ through DS_{N}), calculate at least one “feature” for each data source node based on the received data, and “automatically” output a correction or manipulation for execution by a controller of the medical device to maintain the operation of the medical device in a normal operating state or to return the operation of the medical device to the normal operating state, as well as to output an alert signal to one or more remote monitoring devices 170 when appropriate (e.g., for display to a user). According to some embodiments, the alert signal might be transmitted to a device controller, a system controller, a Human-Machine Interface (“HMI”), or to a user (e.g., patient, non-patient medical device operator) via a number of different transmission methods. Note that one receiver of the alert signal might be a cloud database. The alert signal may indicate to the recipient that the medical device is experiencing, or will be experiencing, a fault, or a cyber-attack (“compromise”).

As used herein, the term “feature” may refer to, for example, mathematical characterizations of data. Examples of features as applied to data might include the maximum, minimum, mean, standard deviation, variance, range, current value, settling time, Fast Fourier Transform (“FFT”) spectral components, linear and non-linear principal components, independent components, sparse coding features, deep learning features, etc. Moreover, the term “automatically” may refer to, for example, actions that can be performed with little or no human intervention.

As used herein, devices, including those associated with the system 100 and any other device described herein, may exchange information via any communication network which may be one or more of a Local Area Network (“LAN”), a Metropolitan Area Network (“MAN”), a Wide Area Network (“WAN”), a proprietary network, a Public Switched Telephone Network (“PSTN”), a Wireless Application Protocol (“WAP”) network, a Bluetooth network, a wireless LAN network, and/or an Internet Protocol (“IP”) network such as the Internet, an intranet, or an extranet. Note that any devices described herein may communicate via one or more such communication networks.

The offline decision manifold creation module 140 may store information into and/or retrieve information from various data stores, such as the normal space data source 110 and/or the abnormal space data source 120. The various data sources may be locally stored or reside remote from the offline decision manifold creation module 140 (which might be associated with, for example, offline or online learning). Although a single offline decision manifold creation module 140 is shown in FIG. 1, any number of such devices may be included. Moreover, various devices described herein might be combined according to embodiments of the present invention. For example, in some embodiments, the offline decision manifold creation module 140 and one or more data sources 110, 120 might comprise a single apparatus. The offline decision manifold creation module 140 functions may be performed by a constellation of networked apparatuses, in a distributed processing or cloud-based architecture.

A user may access the system 100 via one of the monitoring devices 170 (e.g., a Personal Computer (“PC”), tablet, or smartphone) to view information about and/or manage abnormal information in accordance with any of the embodiments described herein. In some cases, an interactive graphical display interface may let a user define and/or adjust certain parameters (e.g., abnormal state detection trigger levels) and/or provide or receive automatically generated recommendations or results from the offline decision manifold creation module 140 and/or abnormal state detection, prediction and correction module 150.

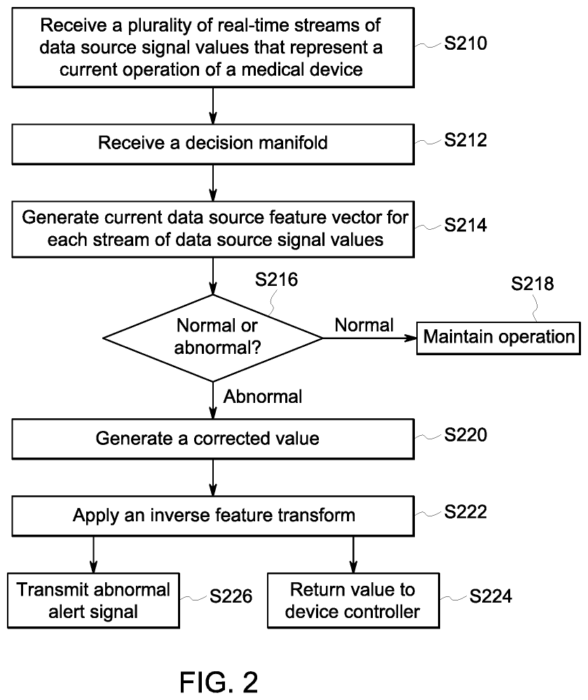

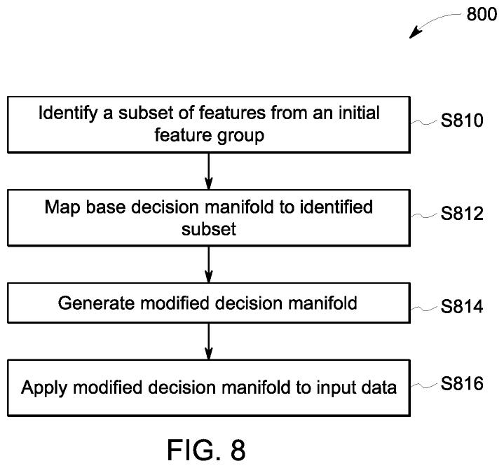

For example, FIG. 2 illustrates a process that might be performed by some or all of the elements of the system 100 described with respect to FIG. 1. Process 200, and any other process described herein (e.g., process 800 in FIG. 8), may be performed using any suitable combination of hardware (e.g., circuit(s)), software or manual means. For example, a computer-readable storage medium may store thereon instructions that when executed by a machine result in performance according to any of the embodiments described herein. In one or more embodiments, the system 100 is conditioned to perform the process 200/800 such that the system is a special-purpose element configured to perform operations not performable by a general-purpose computer or device. Software embodying these processes may be stored by any non-transitory tangible medium including a fixed disk, a floppy disk, a CD, a DVD, a Flash drive, or a magnetic tape. Examples of these processes will be described below with respect to embodiments of the system, but embodiments are not limited thereto. The flow charts described herein do not imply a fixed order to the steps, and embodiments of the present invention may be practiced in any order that is practicable.

Initially, at S210, a plurality of real-time heterogeneous data source nodes 110/120 and the module 150 may receive streams of data source node signal values (“data”) over time that represent a current operation of a medical device. At least one of the data source nodes (e.g., controller nodes, etc.) may be associated with, for example, sensor data, a control intermediary parameter, and/or a control logic value. As described further below, the source nodes 110/120 may receive the data over time and transmit it to the creation module 140 to create the manifold. In one or more embodiments, the data received by the abnormal state detection, prediction and correction module 150 is raw data. It is noted that the use of raw data, as compared to filtered data, in the transformation process described below, provides for a more defined distinction between normal and abnormal operating spaces.

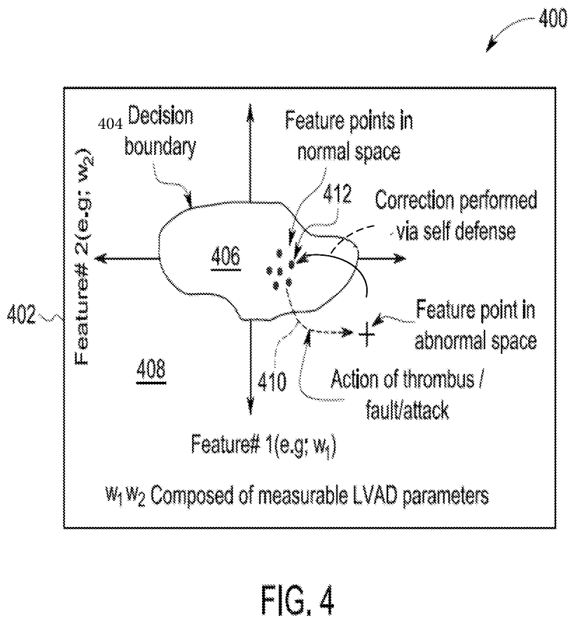

At S212, the abnormal state detection, prediction and correction module 150 receives a decision manifold 400. The decision manifold 400 may separate a normal operating space 406 from an abnormal operating space 408 for a particular source, as described further below with respect to FIG. 4.

Next, at S214, the abnormal state detection, prediction and correction module 150 performs a feature extraction process on each stream of data to generate at least one feature vector. According to some embodiments, at least one of the feature vectors is associated with principal components, statistical features, deep learning features, frequency domain features, time series analysis features, logical features, geographic or position-based locations, and/or interaction features.

The generated feature vector may then be compared to a corresponding decision manifold 400 (e.g., a linear boundary, non-linear boundary, multi-dimensional boundary, etc.) for that data source node in substantially real-time to determine in S216, via the abnormal state detection and/or prediction model 151, 152 whether the feature vector maps to the normal operating space or the abnormal operating space.

According to some embodiments, at least one data source node is associated with a plurality of multi-dimensional decision boundaries and the comparison at S216 is performed in connection with each of those boundaries. As used herein, the terms “decision boundary” and “decision manifold” may be used interchangeably. Note that a decision boundary might be generated, for example, in accordance with a feature-based learning algorithm and a high-fidelity model or a normal operation of the medical device. In one or more embodiments, the abnormal state detection or prediction models 151, 152 associated with a decision boundary may, according to some embodiments, be dynamically obtained and adapted based on a transient condition, a steady state model of the medical device, and/or data sets obtained while operating the system as in self-learning systems from incoming data stream.

In one or more embodiments, in addition to determining whether the feature vector is in the normal or abnormal operating space with the detection model 151, the prediction model 152 may determine whether there is a possibility that the feature vector will be in the normal or abnormal operating space (“prediction”). For example, the abnormal state detection, prediction and correction module 150 may execute a dynamic forecasting model 152 (“prediction model”) representing time-evolution of features in a state variable form. The prediction model 152 may be used in real-time to detect the possibility of malfunction of the medical device by projecting the time evolution of the features into a future time horizon and determining when the path is likely to intercept the decision manifold 400 and determining when the path is likely to intercept the decision manifold 400. The prediction model may output the prediction of the feature vector a few time steps ahead. The inventors note that this may be helpful such that the corrected value, described below, may be enabled prior to the medical device malfunction to prevent the feature vector from crossing the manifold from normal operating space to abnormal operating space. In one or more embodiments, the detection model 151 and the prediction model 152 may be execute simultaneously, substantially simultaneously or in any suitable order. It is noted that the simultaneous or substantially simultaneous execution thereof may serve as an early warning and save valuable time in neutralizing a harmful attack.

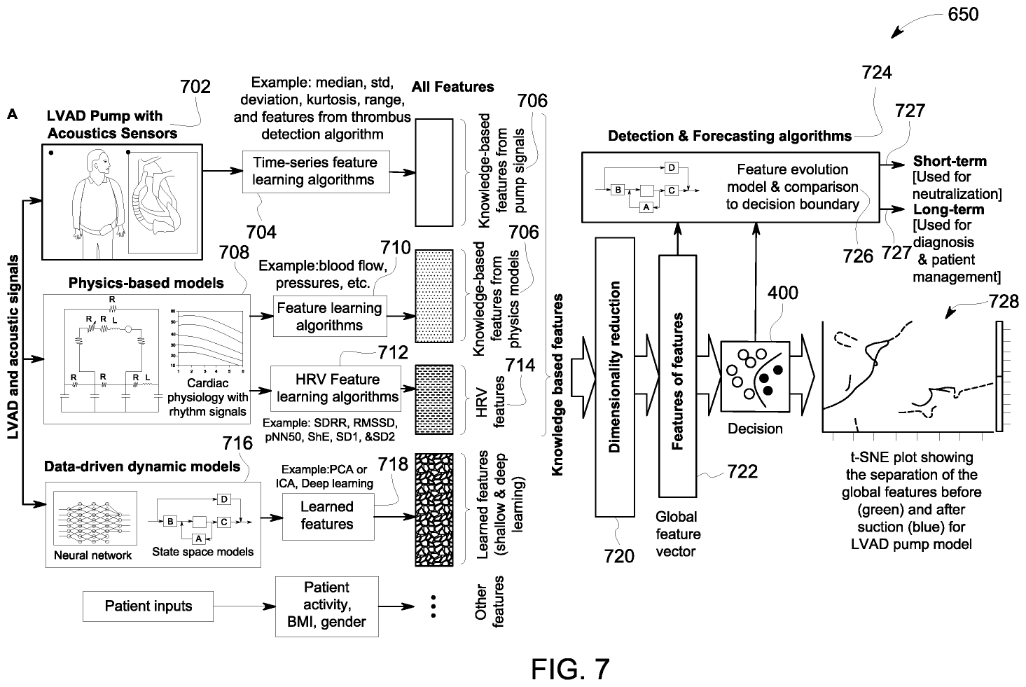

In one or more embodiments, parameters for each prediction model 152 may be learned separately by the abnormal state detection, prediction and correction module 150 from the training data sets. Continuing with the non-exhaustive example, the physics-based model may contain access to individual parameters, such as intracardiac hemodynamics. It is noted the physics-based model may be part of the feature evolution model, the data generation and feature discovery processes, described in FIGS. 1 and 7. The abnormal state detection, prediction and correction module 150 may extract Heart Rate Variability (HRV) metrics in time and frequency domain, to learn the bounds on these parameters. HRV is a physiological quantity representing the state of the autonomous nervous system. Heart rate and pump flow signals may be used to control pump speed to automatically avoid the occurrence of suction, for example, which is undesirable.

Turning back to the process 200, when, at S216, the feature vector maps to the normal operating space 406, the process 200 proceeds to S218, where the operating state of the medical device is maintained for those features, and the process 200 ends.

When, at S216, the feature vector maps to the abnormal operating space 408, the process 200 proceeds to S220, and the correction model 153 of the abnormal state detection, prediction and correction module 150 generates a corrected value (“e.g., neutralized value”) for the feature vector to map the feature vector to the normal operating space 406, which may neutralize the effect of the abnormalities. In one or more embodiments, with the detection model 151 and forecasting model 152 executing in parallel, the correction model 153 may find an estimated corrected value for the feature vector by solving a boundary constrained optimization problem to map the global features (i.e., features of features) inside the decision boundary. The solution to the optimization problem may provide the corrected value. In one or more embodiments, a function (s) i.e. decision boundary or decision manifold may be calculated whereby if the function is negative, the feature is in a normal operating space, and if the function is positive, the feature is in an abnormal operating space.

In one or more embodiments, the boundary constrained optimization problem may be to minimize ∥wₖ−w₀−Φg∥ₗ_{₀} subject to s(g)<0, where wₖ is the first level feature vector obtained by stacking data specific to a data node at a given time instance k; and w₀ is a centroid of the feature vectors (obtained offline), g is the global feature vector (the second level feature vector after data reduction), l₀ denotes the zero-norm of the vector and s(g) is a function of the global feature vectors and represents the decision manifold or boundary 400, etc.

In one or more embodiments, the global feature may be mapped to a location where normal activity is centered, or to any other location within the decision boundary. It is noted that mathematically, imposing the decision boundary as a constraint during the estimation precure may provide that the estimated true features (e.g., for LPVD, signals such as pump speed, power, flow) lie in the normal operating space. The optimization may be NP-hard and may introduce additional complexity because it may generally be non-convex. The optimization problem may be solved with Boundary and Performance Constrained Resilient Estimators, Boundary Kernel Resilient Estimators, and any other suitable technique.

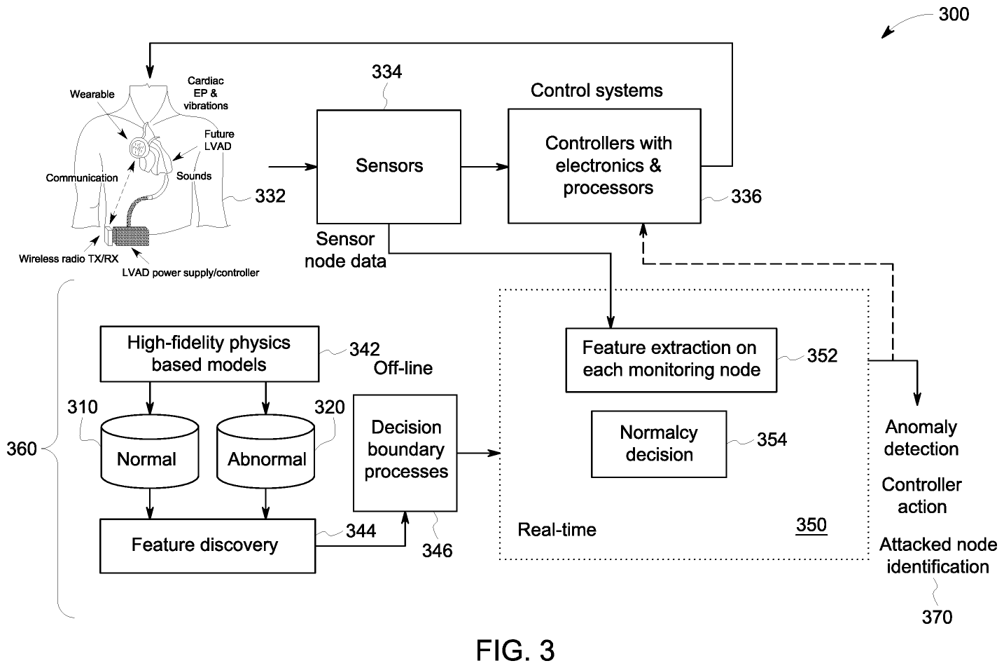

In one or more embodiments, after the optimization problem is solved in S220, an inverse feature transform may be applied in S222 to the output of the optimization problem to generate real-time corrected signals to be fed to the controller. The inverse feature transform may invert the feature vector mapping to the abnormal operating space. It is noted that in cases where the inverse exists (e.g., Principal Component Analysis (PCA)), direct inversion may be used. Else, in cases such as nonlinear PCA, autoencoders—an approximation technique—may be used. Other suitable methods may be used. Next, in S225, the abnormal state detection, prediction and correction module 150 returns the corrected value to a controller 336 (FIG. 3) of the medical device 332 (FIG. 3). The medical device controller, in turn, operates the medical device based on the returned signal such that the medical device operates in normal (e.g., safe) manner via, for example, manipulating the device to maintain the current operation in the event of a predicted malfunction and correct the operation in the event of an executed malfunction and track the desired setpoints preprogrammed by a physician.

In one or more embodiments, the system may also automatically transmit, at S226, an abnormal alert signal (e.g., a notification message, etc.) based on results of the comparisons performed at S216. In one or more embodiments S226 may be performed prior to S220, in parallel with S220, or after S220. The abnormal state might be associated with, for example, a medical device controller attack (“compromise”) (e.g., signals going into (or in some instances out of), the controller), a data source node attack, and/or medical device damage that may or may not require at least one new part. According to some embodiments, one or more response actions may be performed when an abnormal alert signal is transmitted. For example, one or more parameters might be automatically modified, a software application might be automatically triggered to capture data and/or isolate possible causes, etc. Note that an abnormal alert signal might be transmitted via a cloud-based system, such as the PREDIX® field agent system. Note that according to some embodiments, a cloud approach might also be used to archive information and/or to store information about boundaries.

Some embodiments described herein may take advantage of the physics of the medical device and the associated control system by learning a priori from tuned high-fidelity equipment models and/or actual “on the job” data to detect single or multiple simultaneous adversarial threats to, or malfunctions of, the system. Moreover, according to some embodiments, all data source node data may be converted to features using advanced feature-based methods, and the operation of the control system may be monitored in substantially real-time. Abnormalities may be detected by classifying the monitored data as being “normal” or abnormal. This decision boundary may be constructed using dynamic models and may help to enable early detection of vulnerabilities (and potentially avert catastrophic failures) allowing the medical device controller to restore operation in a timely fashion.

Note that an appropriate set of multi-dimensional feature vectors, which may be extracted automatically (e.g., via an algorithm) and/or be manually input, might comprise a good predictor of measured data in a low dimensional vector space. According to some embodiments, appropriate decision boundaries for the decision manifold 400 may be constructed in a multi-dimensional space using a data set which is obtained via scientific principles. Moreover, multiple algorithmic methods (e.g., support vector machines, one of the machine learning techniques) may be used to generate decision boundaries. Since boundaries may be driven by measured data (or data generated from high fidelity models), defined boundary margins may help to create an abnormal zone in a multi-dimensional feature space. Moreover, the margins may be dynamic in nature and adapted based on a transient or steady state model of the device and/or be obtained while operating the system as in self-learning systems from incoming data streams. According to some embodiments, a training method may be used for supervised learning to teach decision boundaries. This type of supervised learning may take into account an operator's knowledge about system operation (e.g., the differences between normal and abnormal operation).

Note that many different types of features may be utilized in accordance with any of the embodiments described herein, including principal components (weights constructed with natural basis sets) and statistical features (e.g., mean, variance, skewness, kurtosis, maximum, minimum values of time series signals, location of maximum and minimum values, independent components, etc.). Other examples include deep learning features (e.g., generated by mining experimental and/or historical data sets) and frequency domain features (e.g., associated with coefficients of Fourier or wavelet transforms). Note that a deep learning technique may be associated with, for example, an auto-encoder, a de-noising auto-encoder, a restricted Boltzmann machine, neural networks etc. Embodiments may also be associated with time series analysis features, such as cross-correlations, auto-correlations, orders of the autoregressive, moving average model, parameters of the model, derivatives and integrals of signals, rise time, settling time, etc. Still other examples include logical features (with semantic abstractions such as “yes” and “no”), geographic/position locations, and interaction features (mathematical combinations of signals from multiple data source nodes and specific locations). Embodiments may incorporate any number of features as required for accurate representation of the data and the interplay between different data nodes.

Thus, some embodiments may provide an advanced anomaly detection and correction process to detect cyber-attacks on, or malfunctions with, for example, medical devices. The process may identify which signals(s) are abnormal using data source node-specific decision boundaries and may inform a control system to take corrective actions.

An abnormality detection algorithm may process a real-time medical device signal data stream and then compute features (multiple identifiers) which can then be compared to the sensor specific decision boundary. A block diagram of a system 300 utilizing a sensor specific medical device abnormality detection algorithm according to some embodiments is provided in FIG. 3. In particular, a medical device 332 provides information to sensors 334 which helps controllers with electronics and processors 336 adjust operation of the medical device 332. An offline abnormal state detection system 360 may include one or more high-fidelity physics-based models 342 associated with the medical device 332 to create normal data 310 and/or abnormal data 320. The normal data 310 and abnormal data 320 may be accessed by a feature discovery component 344 and processed by decision boundary process 346 while off-line (e.g., not necessarily while the medical device 332 is operating). The decision boundary process 346 may generate decision boundaries for various data source nodes. Each decision boundary may separate the data set into two data sets in the feature space which is constructed by running a binary classification algorithm, such as a support vector machine using the normal data 310 and abnormal data 320 for each data source node signal (e.g., from the sensors 334, and/or controllers 336).

An abnormality platform 350 may receive the boundaries along with streams of data from the data source nodes. The platform 350 may include a feature extraction on each data source node element 352 and a normalcy decision 354 with a process to detect abnormalities in individual signals using sensor specific decision boundaries. The platform 350 may generate outputs 370, such as an anomaly decision indication (e.g., abnormal alert signal), and/or a corrected value for a controller action.

During real-time detection and correction, contiguous batches of data source node data may be processed by the platform 350, and the feature vector extracted. The location of the vector for each signal in high-dimensional feature space may then be compared to a corresponding decision boundary. If it falls within the abnormal operating space, then a malfunction may be declared. The data may be corrected such that the vector may be moved to the normal region. The corrected vector is input back to the controller of the medical device, and the medical device continues operation. When the system again determines the same feature is associated with an abnormal feature vector within a user-defined period of time, the system may determine the medical device has a fault that may need to be repaired or corrected. When the abnormal feature vector does not return within the user-defined period of time, the system may determine the medical device has been attacked. This may be done by individually monitoring, overtime, the location of the feature vector with respect to the decision boundary.

According to some embodiments, it may be detected whether or not a signal is in the normal operating space (or abnormal space) through the use of localized decision boundaries and real time computation of the specific signal features.

FIG. 4 illustrates a decision manifold 400, including boundaries and a feature vector that may be associated with data source node parameters in accordance with some embodiments. In particular, a graph 402 includes a first axis representing value weight 1 (“w1”), a feature 1, and a second axis representing value weight 2 (“w2”), a feature 2. Values for w1 and w2 might be associated with, for example, outputs from a Principal Component Analysis (“PCA”) that is performed on the input data. PCA may be one of the analyses that may be used by the process to characterize the data, but note that other analyses may be leveraged.

The graph includes a decision boundary 404. The space within the decision boundary (e.g., shaded region), may be the normal operating space 406. The space outside of the decision boundary may be the abnormal operating space 408. The graph also includes an indication associated with current feature location for feature points in the normal operating space 406 (illustrated with a “circle” on the graph), and an indication associated with current feature location for feature points in the abnormal operating space 408 (illustrated with a “+” on the graph). As indicated on the graph 400 by arrow 410, an action of fault or attack (e.g., resulting in thrombus) may move the location of the feature point from the normal operating space 406 to the abnormal operating space 408. The graph 400 also indicates, by arrow 412, that per a correction performed by the abnormal state detection, prediction and correction module 150, the location of the feature point may be moved from the abnormal operating space 408 to the normal operating space 406. In one or more embodiments, the system 100 may determine the operation of the medical device 332 is normal or abnormal based on the location of the feature point in the decision manifold 400.

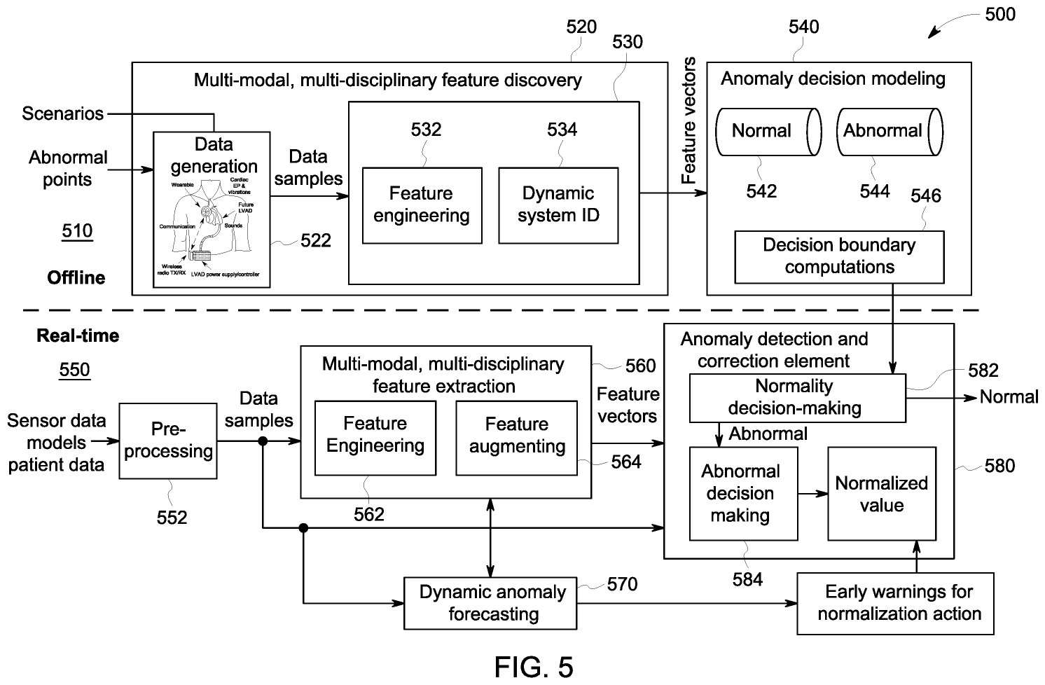

FIG. 5 is an offline and real-time anomaly detection and prediction tool 500 according to some embodiments. In particular, the architecture 500 includes an offline portion 510 (e.g., that performs calculations once every user-defined amount of time) and a real-time portion 550. The offline portion 510 includes a Multi-Model, Multi-Disciplinary (“MMMD”) feature discovery element 520 that receives scenarios and abnormal points. The scenarios and abnormal points may, for example, be provided to a data generation element 522 (e.g., associated with a medical device model) that generates data samples that are provided to feature engineering 532, dynamic system identification 534, and/or feature augmenting elements of a feature discovery element 530 that in turn provides feature vectors to an anomaly decision modeling system 540. The anomaly decision modeling system 540 may include normal data 542 and abnormal data 544 that are used, along with the received feature vectors, by decision boundary computations 546 to output feature boundaries to an anomaly detection and correction element 580 in the real-time portion 550 of the architecture 500.

The real-time portion 550 of the architecture 500 may also include a pre-processing element 552 that receives information from homogeneous sources, such as sensor data, patient/user inputs (activity, BMI, gender, etc.), acoustic signals, medical device power, flow, etc., etc. The pre-processing element 552 may then generate data samples that are provided to a MMMD feature extraction unit 560 and a dynamic anomaly forecasting and situation awareness element 570 (e.g., to generate early warnings). The feature extraction unit 560 might include, for example, feature engineering 562 and feature augmenting 564, and provide feature vectors to the anomaly detection and correction element 580. According to some embodiments, the anomaly detection and correction element 580 includes normality decision making 582 (e.g., to generate a normal indication) and abnormal decision making 584 (e.g., to generate abnormal indications, etc.).

According to some embodiments, the architecture 500 may implement a proposed framework that consists of two steps: (1) a feature-based model-assisted learning approach 510 for use in offline computation; and (2) real-time, high speed detection process 550 (e.g., operating from approximately once every second to once every minute) that leverages heterogeneous data sources. The offline decision boundary tool 510 may use a physics-based medical device model (e.g., associated with the data generation element 522) to characterize different operation points as normal or abnormal conditions. The real-time tool 550 may use the decision boundary, various mapping functions built during the offline process 510 and real-time data from heterogeneous sensors to identify abnormal conditions from normal operation of the system and correct (“normalize”) the values associated with the abnormal condition to result in a normal operation and indication thereof.

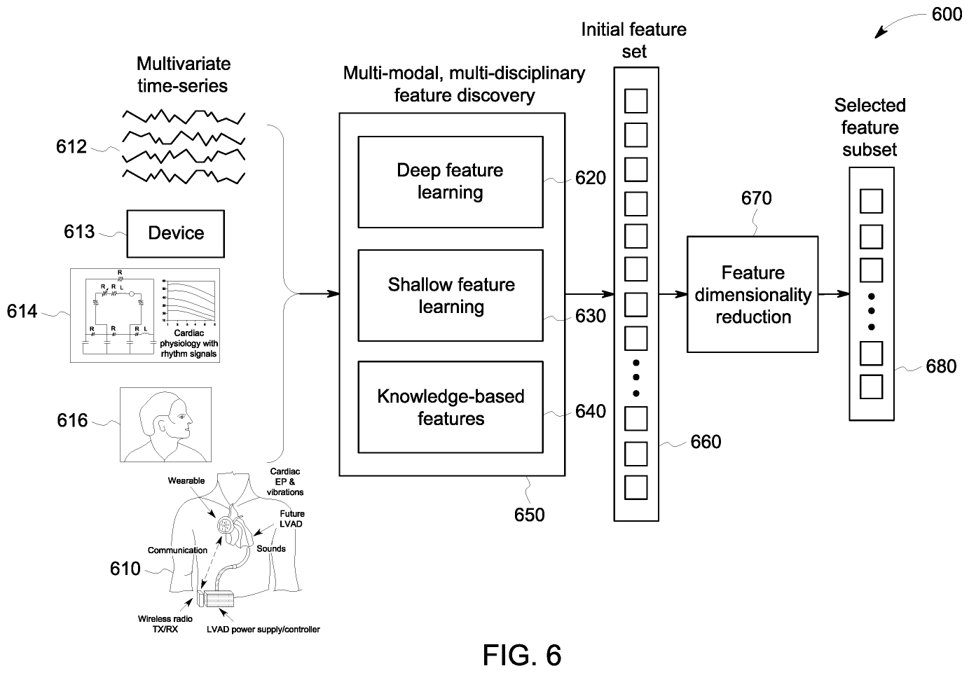

Note that in the framework described with respect to FIGS. 5 and 6, identifying salient features may be an important aspect of developing control optimization for dynamic systems as well as machine learning and data mining solutions. Extracting features from different data sources (e.g., time-series sensor measurements, device data, models, patient data, etc.) is a way of leveraging information from different types of data sources (multiple “modalities”) for improved performance. According to some embodiments, an MMMD feature discovery framework may generate features of features from different data sources. That is, in an integrated framework an initial vector of static features may be extracted (e.g., using machine learning techniques). Then, in order to capture the evolution of features over time, a dynamic model may be identified for an optimal subset of the original features, and dynamic model features (or “features of the features”) may be extracted to be augmented as the overall feature vector. Note that features might be associated with a dynamic model comprising, for example, stability margins, controllability indices, observability indices, elements of an observability matrix, elements of a controllability matrix, poles, and/or zeros of the dynamic model of the evolution of features over time.

FIG. 6 is a feature vector information flow diagram 600 wherein a heterogeneous set of data sources are associated with a medical device 610. In the non-exhaustive example shown herein, the medical device is an LVAD. The flow diagram may apply to other suitable medical devices. The data sources might include, for example, sensor information 612 (e.g., acoustic or other signals from sensor nodes), device information 613 (e.g., pump power, flow, pulsatility index), models 614, and patient data 616, etc. Information from the data sources 612, 613, 614, 616 is provided to MMMD feature discovery 650 which generates an initial feature set 660. The MMMD feature discovery 650 might include, according to some embodiments, deep feature learning 620, shallow feature learning 630, and/or knowledge-based features 640. Because the initial feature set 660 might be relatively large, a feature dimensionality reduction process 670 may be utilized to create a selected feature subset 680.

Given the heterogeneous data types, the system may extract features from each individual data source using different feature extraction methods and then combine the results to create the initial feature set 660 (this “combining” process is often referred as “feature fusion” in machine learning and data-mining domains). Because the initial feature set 660 is likely substantially large, the system then applies feature dimensionality reduction 670 techniques to reduce the number of features to a reasonable level before the selected feature subset 680 is used by an anomaly detection engine. The reduction may provide a better separation between abnormal and normal operating spaces. It is noted that feature reduction may include extracting successively deep levels of features. With each successive level of extraction, the level may lose specificity with respect to a particular signal, but may better describe the relationships between multiple features. For example, an initial feature set (1′ level) may include signals with physical significance (e.g., BMI, gender, heart rate, power to device, etc.) but at a higher level, the feature may be a correlation or distance between signals or some statistical quantity like mean, max, median. Second and third levels may be groups of physical features together (e.g., BMI*2/heartrate+square root of blood pressure).

Note that the MMMD feature discovery 650 may use physics, physiology and machine learning with knowledge-based feature 640 engineering, shallow feature learning 630, and deep feature learning 620. Knowledge-based feature 640 engineering may use domain or engineering knowledge of the medical device and it's associated condition (e.g., LVAD and the circulatory system) 610 physics to create features from different sensor measurements. These features might simply be statistical descriptors (e.g., maximum, minimum, mean, variance, different orders of moments, etc.) calculated over a window of a time-series signal and its corresponding Fast Fourier Transformation (“FFT”) spectrum as well.

With respect to the LVAD and circulatory system, the knowledge-based features 640 might also utilize time-domain heart rate variability (HRV) data which is a physiological quantity representing the state of autonomous nervous system. A parameterized dynamic model of pump dynamics may provide knowledge-based domain level features related to blood flow variations, circadian rhythm, pump power, patient age, BMI and other biomarkers correlated to the pump speed. Knowledge-based features related to medical devices may also include patient data, such as diet, physical activities, medication and supplements, that may be collected from patient's daily check lists, for example. These machine-learning features may be incorporated in the feature vector during learning and then in real-time use.

Although knowledge-based feature 640 engineering is a traditional approach for feature extraction, it is often a laborious, manual process. The approach is also very application specific, and therefore not generalizable or scalable. Learning features directly from data (e.g., via machine learning) may address these issues. Data-driven feature learning involves both shallow learning and deep learning. For example, shallow feature learning 630 techniques include many unsupervised learning (e.g., k-means clustering), manifold learning and nonlinear embedding (e.g., isomap methods and Locally-Linear Embedding (“LLE”)), low-dimension projection (e.g., Principal Component Analysis (“PCA”) and Independent Component Analysis (“ICA”)), and/or neural networks (e.g., Self-Organizing Map (“SOM”) techniques). Other examples of shallow feature learning 630 techniques include genetic programming and sparse coding. The deep feature learning 620 may represent a sub-field of machine learning that involves learning good representations of data through multiple levels of abstraction. By hierarchically learning features layer by layer, with higher-level features representing less specific aspects of a signal feature data, deep feature learning 620 can discover sophisticated underlying structure and features.

To build the decision manifold 400, first a feature extraction process is performed with data streams from different sources and the features are then stacked to form local feature vectors in an initial feature set (level 1 features). The multi-modal, multi-disciplinary feature discovery 650 (or “extraction”) will most likely lead to a large number of features in the initial feature set 660. As a non-exhaustive example, the initial set may include 100 features, some of which may be redundant. Directly using such a large number of features may be burdensome for down-stream anomaly detection models. The local feature vectors (e.g., initial feature set) are then stacked into one big vector, on which further dimensionality reduction is carried out to obtain what is referred to as higher-level feature vector (i.e., feature of features) or global feature vector. Feature dimensionality reduction 670 may reduce the number of features by removing redundant information and finding patterns in the data while maximally preserving useful information of the features. Embodiments of feature dimensionality reduction described herein may be associated with feature selection and/or feature transformation techniques. The global features (“feature of features”) may capture the interplay between different variables and their corresponding features in this higher dimensional space than in the original time domain/space. The global feature vector may then be marked as normal or abnormal based on its signed distance from the multi-modal decision manifold. Continuing with the non-exhaustive example, the 100 features in the initial feature set may be reduced to five features as the features of features. In one or more embodiments, the reduction process 670 may iterate until the number of features is reduced to a pre-defined number such that the reconstruction of the original data stream from the features achieves a predefined level of accuracy.

In one or more embodiments, the MMMD feature discovery 650 may perform a feature dimensionality reduction process to generate a selected feature vector subset. In one or more embodiments, the MMMD feature discovery 650 may be used to calculate and output at least one decision boundary for an abnormal detection model based on the selected feature vector subset. According to some embodiments, the selected feature vector subset is further used in connection with anomaly detection, anomaly correction, anomaly forecasting, and/or system diagnosis.

By combining knowledge-based feature 650 engineering and advanced deep feature learning 620 techniques (and applying those to different data sources), the MMMD feature discovery 650 framework may be effective in discovering a feature set that provides accurate and reliable anomaly detection. Note that the framework is generic (and can be used effectively for other analytics applications) and flexible in handling situations where the numbers and the types of available data sources vary from system to system.

FIG. 7 shows a non-exhaustive example of the MMMD feature discovery framework 650 and feature of features learning using continuous streams of data from a patient's LVAD pump and models 610. For example, as shown herein, the data may be received from signals from the LVAD pump with acoustic sensors 702, and a time-series feature learning algorithm 704 may extract knowledge-based features 706 therefrom (e.g., median, standard deviation, kurtosis, range, and features from a thrombus detection algorithm, etc.). The data received from the signals from the LVAD pump with acoustic sensors 702 may also be input to physics-based models 708, and a feature-learning algorithm 710 may extract knowledge-based features (e.g., blood flow, pressure, etc.) 706. Also, an HRV feature learning algorithm 712 may extract HRV features 714 (e.g., standard deviation of RR-interval or inter-beat interval (SDRR), root mean square of successive differences (RMSSD), percentage of adjacent NN intervals that differ from each other by more than 50 ms (pNN50), ShE, S01, and SD2) from the data input to the physics-based models 708. The data received from the LVAD pump with acoustic sensors 702 may also be input to data-driven dynamic models 716, and one or more learned features 718 (shallow (PCA or ICA) and deep learning) may be extracted. It is noted that the physics-based model and the data-driven model may be tuned to the patient data in that the parameters specific to the patient (e.g. diet, medication, heart rate etc.) are identified and implemented such that the model is the best representation of the patient's physiology. Additionally, the framework 650 may receive patient inputs (e.g., patient activity, BMI, gender, etc.), from which other features may be extracted. As described above, there may be too many features, and a feature dimensionality reduction process 720 may reduce the number of features. The reduced feature set is the feature of features (“global feature vector”) 722.

In one or more embodiments, the global feature vector 722 may be input to the dynamic anomaly detection and forecasting element 724, which may include a feature evolution model 726 that predicts or forecasts the global features over a short/long time horizon, and a comparison to a decision manifold. As described above, the dynamic anomaly detection and forecasting element 724 may use the global feature vector 722 for two different time scales, namely short-term (seconds ahead), and long term (hours ahead). The forecasted global features may be the anticipated time-evolution of features, assuming the operational settings for the device remain unchanged. The short-term detection 727 is used for enabling neutralization of an anticipated anomaly, and the long-term detection 727 may be used for diagnosis and patient care. In one or more embodiments, parameters of the time-evolution model may be obtained via 1. Running the tuned hybrid model for a variety of time-based fault/attack scenarios for a predetermined length of time (seconds to hours) and then 2. Performing system identification techniques to map the current features to the future feature vectors. In one or more embodiments, the tuned hybrid model may be used for feature discovery and decision manifold training during the offline phase, while the feature evolution model may be used to understand how the features evolve with time. The hybrid model may, however, allow some insight into how the features evolve with time. The projected time when the global feature vector intersects the multi-dimensional decision boundary is used to predict anomalies and generate early warning at different time scales.

The global feature vector 722 may also be input to the decision manifold 400. In one or more embodiments, the global feature vector 722 may be input to the decision manifold, the output of which may indicate the feature falls into a normal operating space or an abnormal operating space. The decision manifold may be presented to a user, via the remote monitoring devices 170, on a t-SNE plot 728, for example, which may show the separation of the global features before (green) and after suction (blue) for the LVAD pump model.

FIG. 8 illustrates a process 800 for modifying a decision manifold 400, according to one or more embodiments. In one or more embodiments, the decision manifold 400 may be tailored to a specific category of patients, for example, or other grouping. It is noted that a single universal decision manifold may be unlikely to yield accurate detection and forecasting performance across multiple medical devices and patient groups. To resolve this, one or more embodiments provide for the modification of a base decision manifold for different types of devices (e.g., axial and centrifugal pumps) and/or different patient groups (e.g., male/female arrhythmias), or any other suitable groupings. Another non-exhaustive example of a modified decision manifold may be when there are operational differences between destination to transplant (DT) and bridge to transplant LVADs.

To personalize the decision boundary to suit individual patients, such that the model is the best representation of the patient's physiology, initially, at S810, a subset of the features from the initial feature group is identified, wherein the features are specific to the patient. The subset may be identified by a transfer learning process or via any other suitable process. As a non-exhaustive example, the patient may be in the thrombosis group, and as such may be associated with spectral features of the acoustical signals corresponding to pump thrombosis. In S812, the base decision manifold is mapped to the subset group (e.g., patients with thrombosis). In one or more embodiments, the transfer learning process may also identify a function that will perform the mapping described in S812. Other suitable mapping processes may be used. For example, the l1-norm support vector machine (SVM) may be used as the base decision manifold, and it may be adapted to suit the transfer learning process for modifying decision manifolds. After mapping the base decision manifold to the subset group, a modified decision manifold is generated in S814. Next, in S816, the modified decision manifold may be applied to input data to generate alerts when a feature for the individual patient has crossed into the abnormal operating space and/or by using time-evolution models (i.e., forecasting models) of global features and projected time to intersect the modified decision boundaries.

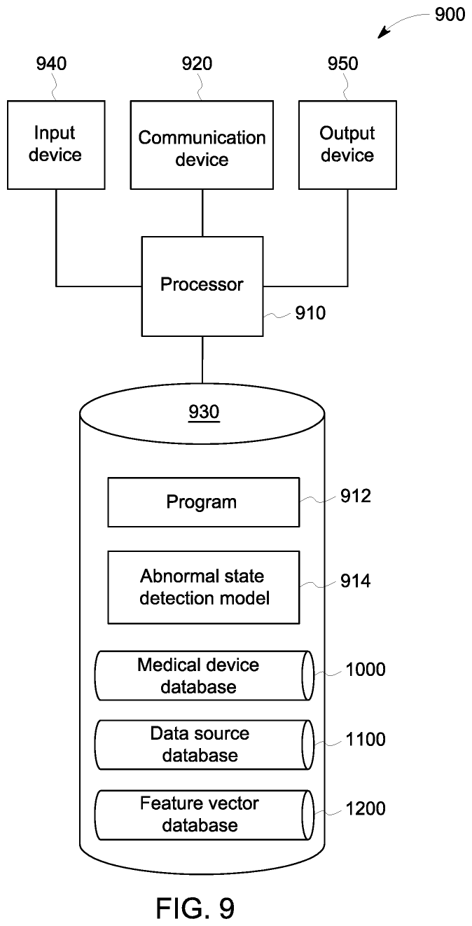

The embodiments described herein may be implemented using any number of different hardware configurations. For example, FIG. 9 is a block diagram of a medical device protection platform 900 that may be, for example, associated with the system 100 of FIG. 1. The medical device protection platform 900 comprises a processor 910, such as one or more commercially available Central Processing Units (“CPUs”) in the form of one-chip microprocessors, coupled to a communication device 920 configured to communicate via a communication network (not shown in FIG. 9). The communication device 920 may be used to communicate, for example, with one or more remote data source nodes, user platforms, etc. The medical device protection platform 900 further includes an input device 940 (e.g., a computer mouse and/or keyboard to input medical device information) and/an output device 950 (e.g., a computer monitor to render a display, provide alerts, transmit recommendations, and/or create reports). According to some embodiments, a mobile device, monitoring physical system, and/or PC may be used to exchange information with the medical device protection platform 900.

The processor 910 also communicates with a storage device 930. The storage device 930 may comprise any appropriate information storage device, including combinations of magnetic storage devices (e.g., a hard disk drive), optical storage devices, mobile telephones, and/or semiconductor memory devices. The storage device 930 stores a program 912 and/or an abnormal state detection, prediction and correction model 914 for controlling the processor 910. The processor 910 performs instructions of the programs 912, 914, and thereby operates in accordance with any of the embodiments described herein. For example, the processor 910 may receive, from a plurality of heterogeneous data source nodes, a series of data source node values over time associated with operation of the medical device control system. The processor 910 may then perform a feature extraction process to generate an initial set of feature vectors. A feature selection process may be performed with a multi-model, multi-disciplinary framework by the processor 910 to generate a selected feature vector subset. At least one decision boundary may be automatically calculated by the processor for an abnormal state detection, prediction and correction model based on the selected feature vector subset. Note that a set of feature vectors might include normal feature vectors and/or abnormal feature vectors. For example, in some cases only normal feature vectors might be used along with unsupervised learning algorithms to construct a decision boundary. In such scenarios, abnormal feature vectors might not be used.

The programs 912, 914 may be stored in a compressed, uncompiled and/or encrypted format. The programs 912, 914 may furthermore include other program elements, such as an operating system, clipboard application, a database management system, and/or device drivers used by the processor 910 to interface with peripheral devices.

As used herein, information may be “received” by or “transmitted” to, for example: (i) the medical device protection platform 900 from another device; or (ii) a software application or module within the medical device protection platform 900 from another software application, module, or any other source.

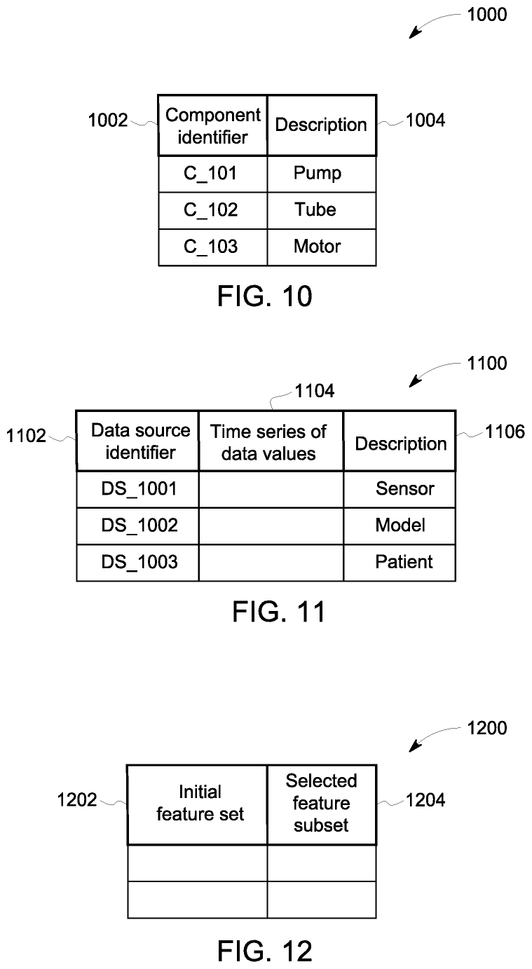

In some embodiments (such as the one shown in FIG. 9), the storage device 930 further stores a medical device database 1000, data source database 1100, and a feature vector database 1200. Example of databases that may be used in connection with the medical device protection platform 900 will now be described in detail with respect to FIGS. 10 through 12. Note that the databases described herein are only examples, and additional and/or different information may be stored therein. Moreover, various databases might be split or combined in accordance with any of the embodiments described herein.

Referring to FIG. 10, a table is shown that represents the medical device database 1000 that may be stored at the medical device protection platform 1000 according to some embodiments. The table may include, for example, entries identifying components associated with a medical device. The table may also define fields 1002 and 1004 for each of the entries. The fields 1002 and 1004 may, according to some embodiments, specify: a component identifier 1002, and description 1004. The medical device database 1000 may be created and updated, for example, off line (non-real time).

The component identifier 1002 might be associated with an element of the medical device and the description 1004 might describe the component (e.g., a pump, a tube, motor, etc.). The medical device database 1000 might further store, according to some embodiments, connections between components (e.g., defining a topology of the device), component statuses, etc. According to some embodiments, the information in the medical device database may be used in connection with knowledge-based features 640 of FIG. 6.

Referring to FIG. 11, a table is shown that represents the data source database 1100 that may be stored at the medical device protection platform 900 according to some embodiments. The table may include, for example, entries identifying data sources associated with a medical device. The table may also define fields 1102, 1104, 1106 for each of the entries. The fields 1102, 1104, 1106 may, according to some embodiments, specify: a data source identifier 1102, a time series of data values 1104, and description 1106. The data source database 1100 may be created and updated, for example, based on information received from heterogeneous sensors.

The data source identifier 1102 may be, for example, a unique alphanumeric code identifying a data source that might provide information to be monitored to protect a medical device. The time series of values 1104 might be associated with a set of numbers being reported by a particular sensor (e.g., representing voltages, currents, etc.) and the description 1106 might describe the type of information being monitored (e.g., from a sensor, model, patient, etc.). The data source database 1100 might further store, according to some embodiments, other information. According to some embodiments, information from the data source database 1100 may be provided as inputs to the MMMD 650 of FIG. 6.

Referring to FIG. 12, a table is shown that represents the feature vector database 1200 that may be stored at the medical device protection platform 900 according to some embodiments. The table may include, for example, entries describing the medical device being analyzed by a MMMD framework. The table may also define fields 1202 and 1204. The fields 1202 and 1204 may, according to some embodiments, specify: an initial feature set 1202, and a selected feature subset 1204. The feature vector database 1200 may be created and updated, for example, offline when a medical device is modified.

The initial feature set 1202 may represent values associated with the initial feature set 660 created by the MMMD feature discovery 650 of FIG. 6. The selected feature subset 1204 may represent values associated with the selected feature subset 680 created by the feature dimensionality reduction 670 of FIG. 6. The selected feature subset 1204 may be used, according to some embodiments, to separate normal behavior from abnormal behavior for a medical device.

The following illustrates various additional embodiments of the invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

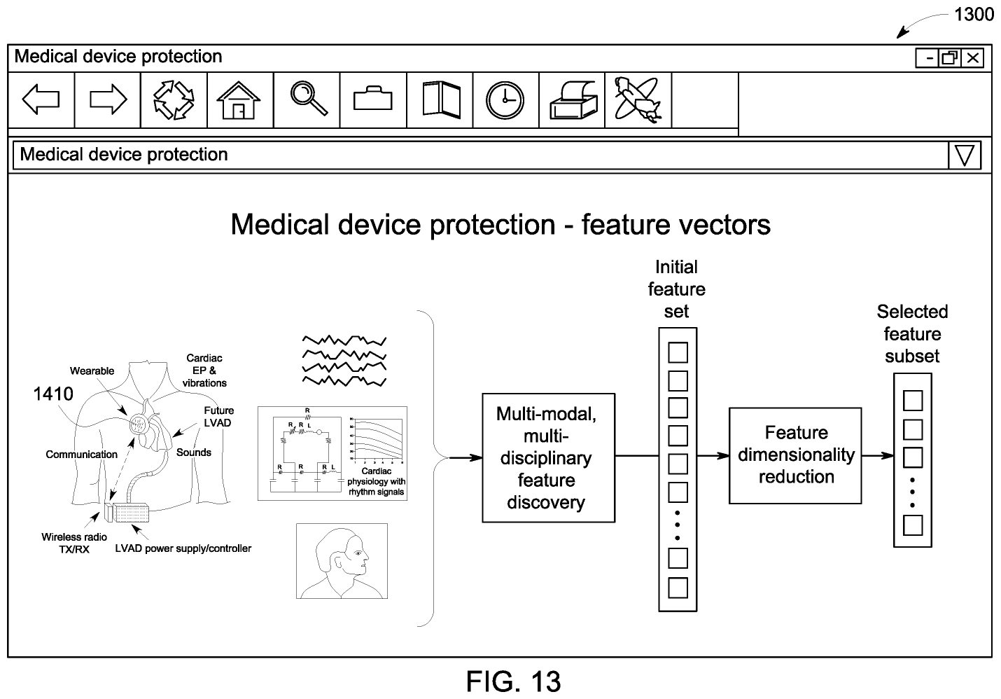

Although specific hardware and data configurations have been described herein, note that any number of other configurations may be provided in accordance with embodiments of the present invention (e.g., some of the information associated with the databases described herein may be combined or stored in external systems). For example, although some embodiments are focused on medical devices, any of the embodiments described herein could be applied to other types of assets, such as damns, wind farms, etc. Moreover, note that some embodiments may be associated with a display of information to an operator. For example, FIG. 13 illustrates an interactive Graphical User Interface (“GUI”) display 1300 that might display information about a medical device 1310 (e.g., including an initial set of feature vectors and a selected feature vector subset).

In addition to automatic threat detection, some embodiments described herein might provide systems with an additional cyber layer of defense and be deployable without custom programming (e.g., when using operating data). Some embodiments may be sold with a license key and could be incorporated as monitoring service. For example, feature vectors and/or boundaries might be periodically updated when equipment in a medical device is upgraded.

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

1. A system to protect a medical device, comprising:
one or more heterogeneous data source nodes generating data associated with operation of the medical device;
an abnormal state detection, prediction and correction module to receive data from one or more heterogeneous data source nodes;
a memory for storing program instructions; and
an abnormal state processor, coupled to the memory, and in communication with the abnormal state detection, prediction and correction module and operative to execute program instructions to:
receive data from one or more heterogeneous data source nodes;
receive a decision manifold separating a normal operating space from an abnormal operating space;
perform a feature extraction process on the received data to generate at least one feature vector;
determine, via the abnormal state detection, prediction and correction module, whether the feature vector maps to the normal operating space or the abnormal operating space in the decision manifold; and
generate, via the abnormal state detection, prediction and correction module, a corrected value for the feature vector to map the feature vector to the normal operating space when it is determined that the feature vector maps to the abnormal operating space.

2. The system of claim 1, wherein the abnormal state processor further comprises program instructions to:
return the corrected value for the feature vector to a controller of the medical device.

3. The system of claim 1, wherein the abnormal state processor further comprises program instructions to:
transmit an abnormal alert signal when it is determined the feature vector maps to the abnormal operating space.

4. The system of claim 1, wherein the abnormal state processor further comprises program instructions to:
determine whether the mapping of the feature vector to the abnormal operation space is based on a fault with the medical device or a compromise of the received data.

5. The system of claim 4, wherein the fault with the medical device or the compromise of the received data is associated with at least one of: (i) a data source node attack, (ii) medical device damage requiring at least one new part.

6. The system of claim 1, wherein the abnormal state processor further comprises program instructions to:
perform a feature dimensionality reduction process to reduce an amount of feature vectors when two or more feature vectors are generated by the feature extraction process prior to determining whether the feature vector maps to the normal operating space or abnormal operating space.

7. The system of claim 6, wherein the feature dimensionality reduction process is associated with a feature transformation technique.

8. The system of claim 6, wherein the feature dimensionality reduction process is associated with a feature selection technique.

9. The system of claim 1, wherein at least one of the heterogeneous data source nodes is associated with at least one of: (i) acoustic data, (ii) patient inputs, (iii) device features; (iv) physics-based models, and (v) data-driven dynamic models.

10. The system of claim 1, wherein the abnormal state processor further comprises program instructions to:
detect a possibility of malfunction of the device by execution of a forecast model, wherein the forecast model outputs the prediction of the feature vector a few time steps ahead;
return the corrected value for the feature vector to a controller of the medical device.

11. The system of claim 1, wherein the decision manifold is generated per a training data set.

12. The system of claim 11, further comprising program instructions to:
modify the decision manifold to correspond to a sub-set of at least one of a device type and a user group.

13. A computer-implemented method to protect a medical device, comprising:
receiving data from one or more heterogeneous data source nodes;
receiving a decision manifold separating a normal operating space from an abnormal operating space;
performing a feature extraction process on the received data to generate at least one feature vector;
determining, via an abnormal state detection, prediction and correction module, whether the feature vector maps to the normal operating space or the abnormal operating space in the decision manifold; and
generating, via the abnormal state detection, prediction and correction module, a corrected value for the feature vector to map the feature vector to the normal operating space when it is determined that the feature vector maps to the abnormal operating space.

14. The method of claim 13, further comprising:
returning the corrected value for the feature vector to a controller of the medical device.

15. The method of claim 13, further comprising:
transmitting an abnormal alert signal when it is determined the feature vector maps to the abnormal operating space.

16. The method of claim 13, further comprising:
determining whether the mapping of the feature vector to the abnormal operation space is based on a fault with the medical device or a compromise of the received data.

17. The method of claim 16, wherein the fault with the medical device or the compromise of the received data is associated with at least one of: (i) a data source node attack, and (ii) medical device damage requiring at least one new part.

18. A non-transitory computer-readable medium storing instructions that, when executed by a computer processor, cause the computer processor to perform a method comprising:
receiving data from one or more heterogeneous data source nodes;
receiving a decision manifold separating a normal operating space from an abnormal operating space;
performing a feature extraction process on the received data to generate at least one feature vector;
determining, via an abnormal state detection, prediction and correction module, whether the feature vector maps to the normal operating space or the abnormal operating space in the decision manifold; and
generating, via the abnormal state detection, prediction and correction module, a corrected value for the feature vector to map the feature vector to the normal operating space when it is determined that the feature vector maps to the abnormal operating space.

19. The medium of claim 18, further comprising instructions to cause the computer processor to perform a method comprising:
returning the corrected value for the feature vector to a controller of the medical device.

20. The medium of claim 18, further comprising instructions to cause the computer processor to perform a method comprising:
determining whether the mapping of the feature vector to the abnormal operation space is based on a fault with the medical device or a compromise of the received data.
